(19) United States
(12) Patent Application Publication
(10) Pub. No.: US 2021/0353912 A1
(43) Pub. Date: Nov. 18, 2021

(54) Body Cavity Access Device

(71) Applicant: AOK Innovations, LLC, Lexington, KY (US)

(72) Inventors: Jon Kiev, Lexington, KY (US); Mindy Phung, Jamaica Plain, MA (US); Henry Stock, Sanbornton, NH (US); Christopher Strahm, Deforest, WI (US)

(21) Appl. No.: 17/302,391

(22) Filed: Apr. 30, 2021

Related U.S. Application Data
(60) Provisional application No. 63/024,290, filed on May 13, 2020.

(51) Int. Cl. A61M 25/06; A61M 1/00; A61M 25/09; A61M 25/00

(52) U.S. Cl. CPC A61M 25/0631; A61M 1/90; A61M 1/67; A61M 2205/586; A61M 25/0097; A61M 25/0606; A61M 25/09

(57) ABSTRACT

A handheld body cavity access device including a housing having a barrel, a handle extending from the barrel, and a cavity. The barrel includes a distal end, a proximal end, and a longitudinal axis. A catheter includes a proximal end coupled to the distal end of the housing, and a distal end spaced away from the housing. A guidewire assembly includes a guidewire and a slide carrying to the guidewire and slidably coupled to the barrel of the housing. The slide is movable relative to the housing between a first guidewire position and a second guidewire position. A needle assembly includes a needle, a needle hub, and a conduit, the needle hub carrying the needle and the conduit within the cavity of the housing. A syringe adapter is disposed in the cavity of the housing and adapted to couple to a syringe tip of a syringe.

### FIELD OF DISCLOSURE

The present disclosure is related to a medical device, and in particular, a handheld medical device for accessing a body cavity.

### BACKGROUND

In the medical field, there are many instances in which a practitioner must access the chest, abdomen, or pelvis, and insert a drainage tube. Examples of these instances include the need to relieve excess fluid in the pericardium, bleeding in the chest cavity, and fluid or air buildup in other body regions due to other medical conditions, trauma, or prior surgery.

In a traditional pericardiotomy, for example, a practitioner must pierce, and drain fluid from, the pericardium, which is a fibrous sac that surrounds the heart. To pierce the pericardium, the practitioner inserts a needle through the chest wall and into the tissue surrounding the heart. The practitioner may need the help of an assistant while inserting the needle to ensure that the needle is not inserted too far and nicks or pierces the heart accidently. Typically in a needle pericardiocentesis (i.e., a procedure where the needle creates a hole in the pericardium), an ultrasound wand is placed on the patient's chest to follow as the needle is placed in the pericardium. Once the needle is in place, the practitioner will use a syringe to aspirate the pericardium. After putting down the syringe, the practitioner will take a guidewire and manually thread the guidewire through the needle. With the guidewire in place, the practitioner may replace the needle with a catheter through the hole in the pericardium created by the needle. In this procedure, the practitioner has the needle, syringe, catheter, and guidewire separately laid out on a table.

In other similar procedures, these steps are taken to access and drain excess fluid from body cavities. If performing in the operating room, the operating doctor often requires assistance to perform the procedure because many medical instruments are used simultaneously to perform each step. These procedures can be cumbersome, involve multiple medical instruments and components, and require coordination with multiple practitioners or healthcare providers to perform the procedures accurately and safely.

### SUMMARY

The present disclosure provides a handheld device that enables a single practitioner to access a body cavity to perform a medical procedure, such as a pericardiotomy. In a first exemplary aspect, a handheld body cavity access device may include a housing, a catheter, a guidewire assembly, a needle assembly, and a syringe adapter. The housing may include a barrel, a handle extending from the barrel, and a cavity. The barrel may include a distal end, a proximal end, and a longitudinal axis extending between the distal and proximal ends of the barrel. The catheter may include a proximal end coupled to the distal end of the housing, and a distal end spaced away from the housing. The guidewire assembly may include a guidewire and a slide carrying the guidewire and slidably coupled to the barrel of the housing. The slide may be movable relative to the housing between a first guidewire position, in which a portion of the slide is spaced away from the housing, and a second guidewire position, in which the guidewire extends through the distal end of the needle. The needle assembly may include a needle, a needle hub, and a conduit. The needle hub may carry the needle and the conduit within the cavity of the housing. The needle hub may be movable between a first needle position, in which a distal end of the needle extends from the distal end of the catheter and a second needle position, in which the needle does not extend from the distal end of the catheter. The syringe adapter may be disposed in the cavity of the housing and may be adapted to couple to a syringe tip of a syringe. The syringe adapter may be coupled to the conduit of the needle assembly.

In a second exemplary aspect, a method of accessing a body cavity using a handheld device is provided. The method may include inserting a needle of a handheld device into a cavity. The handheld device may include a housing having a barrel and a handle extending from the barrel. The barrel may include a distal end, a proximal end, and a longitudinal axis extending between the distal and proximal ends of the barrel. A catheter may include a distal end and a proximal end coupled to the distal end of the housing. The distal end may be spaced away from the housing. A guidewire assembly may include a guidewire and a slide carrying the guidewire and slidably coupled to the barrel of the housing. The method may include inserting the guidewire through a distal end of the needle by sliding the slide relative to the housing between a first guidewire position, in which a portion of the slide is spaced away from the housing, to a second guidewire position, in which the guidewire extends through the distal end of the needle. The method may include retracting the needle of the handheld device by activating an actuation device. The method may also include inserting the catheter into the cavity.

In a preferred form, the catheter, needle, and guidewire may be aligned with the longitudinal axis of the barrel.

In a preferred form, the handle may extend from the barrel at an angle in a range of approximately 30 degrees to 90 degrees relative to the longitudinal axis of the barrel.

In a preferred form, the housing may include a track that is formed in the barrel and is arranged to slidably receive a portion of the slide of the guidewire assembly.

In a preferred form, the housing may include a first half and a second half.

In a preferred form, the first half may include a track to slidably receive a first portion of the slide of the guidewire assembly and the second half may include a track to slidably receive a second portion of the slide of the guidewire assembly.

In a preferred form, a catheter hub may be coupled to the catheter and removably coupled to the distal end of housing.

In a preferred form, a syringe may be disposed within a bore of the handle and coupled to the syringe adapter.

In a preferred form, the syringe may be in fluid communication with the conduit of the needle assembly.

In a preferred form, an actuation assembly may be operatively coupled to the needle assembly to move the needle hub between the first needle position and the second needle position.

In a preferred form, actuation assembly may include a spring and a spring release.

In a preferred form, the spring release may include a first portion disposed outside of the housing and a second portion releasably coupled to the needle hub.

In a preferred form, when the spring release is activated, the second portion of the spring release may disengage from the needle hub to permit the spring to move from a compressed position to an expanded position, thereby moving the needle hub from the first needle position to the second needle position.

In a preferred form, the first portion of the spring release may be a button.

In a preferred from, the second portion of the spring release may be an arm adapted to flexibly engage a latch of the needle hub.

In a preferred form, retracting the needle may include moving a needle hub carrying the needle within the barrel of the housing between a first needle position, in which the distal end of the needle extends from the distal end of the catheter, to a second needle position, in which the distal end of the needle is drawn into the catheter.

In a preferred form, retracting the needle may include releasing a spring to drive the needle hub from the first needle position to the second needle position

In a preferred form, the method may include gripping the handle with a hand.

In a preferred form, inserting the guidewire may include sliding the slide with a thumb of the hand.

In a preferred from, retracting the needle may include pressing a button with a finger of the same hand.

In a preferred form, the method may include removing the proximal end of the catheter from the distal end of the barrel and leaving the distal end of the catheter inserted in the cavity.

In a preferred form, the method may include drawing fluid from the cavity using a syringe coupled to the handle of the housing.

In a preferred form, the syringe may be in fluid communication with the needle.

In a preferred form, the method may include coupling the syringe to a syringe adapter disposed in the housing.

In a preferred form, the syringe adapter may be fluidly coupled to the needle via a conduit.

In a preferred form, the method may include inserting the syringe into a bore of the handle of the handheld device.

### BRIEF DESCRIPTION OF THE DRAWINGS

FIG. 1 is a perspective view of a handheld body cavity access device assembled in accordance with the teachings of the present disclosure, and showing the device in a loaded configuration;

FIG. 2 is a side view of the device of FIG. 1;

FIG. 3 is cross-sectional side view of the device of FIG. 1 in a preloaded configuration and illustrated without a syringe;

FIG. 4 is a cross-sectional view of the device of FIG. 1, taken along the lines 4-4 of FIG. 1;

FIG. 5 is a side view of the device of FIG. 1, illustrating the device in a guidewire placement configuration;

FIG. 6 is a cross-sectional side view of the device of FIG. 5, taken along lines 6-6 of FIG. 5;

FIG. 7 is a cross-sectional side view of the device of FIG. 1, illustrating the device in a configuration prior to needle retraction;

FIG. 8 is a cross-sectional side view of the device of FIG. 1, illustrating the device in a needle retraction configuration; and

FIG. 9 is a perspective, cross-sectional view of the device of FIG. 8.

### DETAILED DESCRIPTION

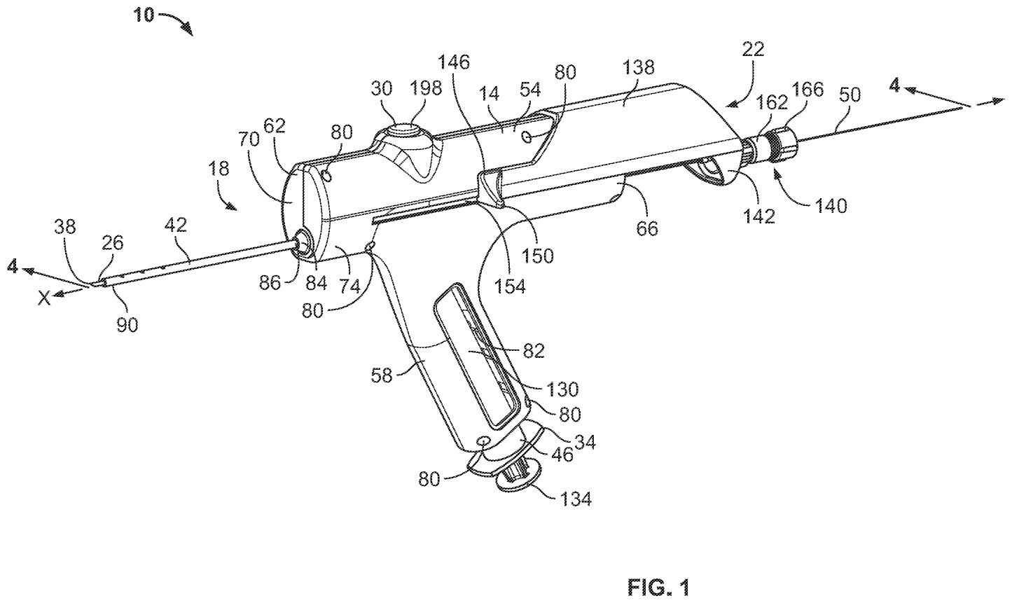

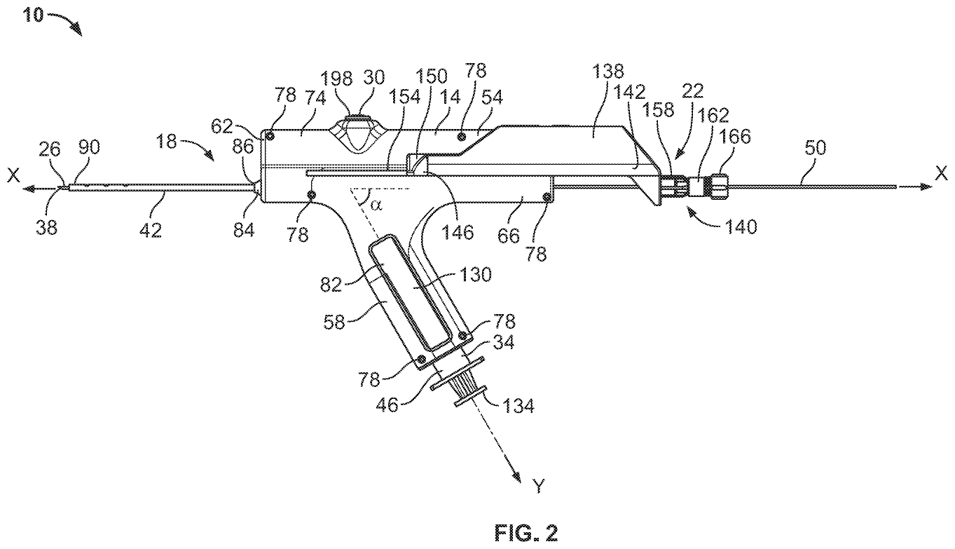

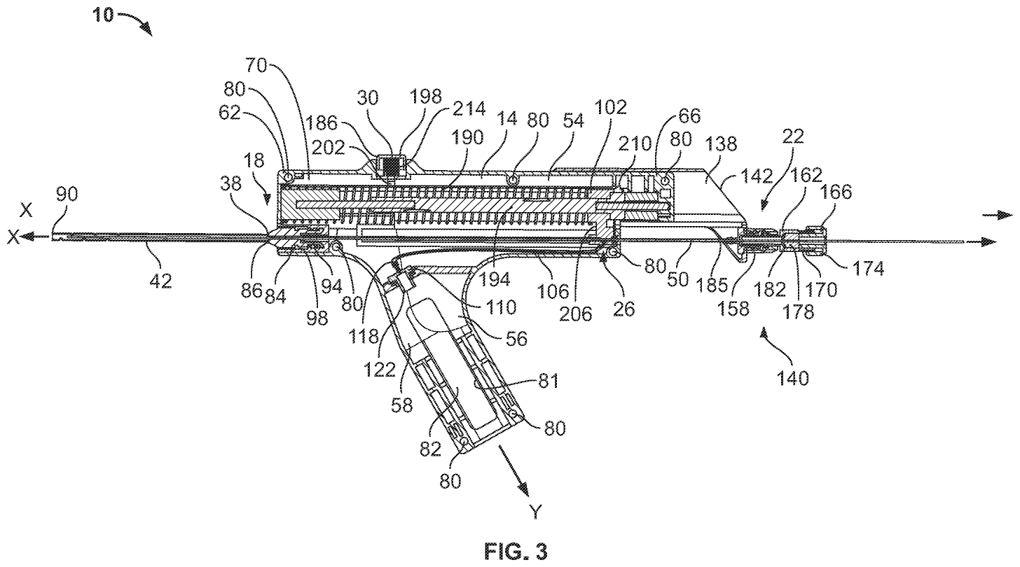

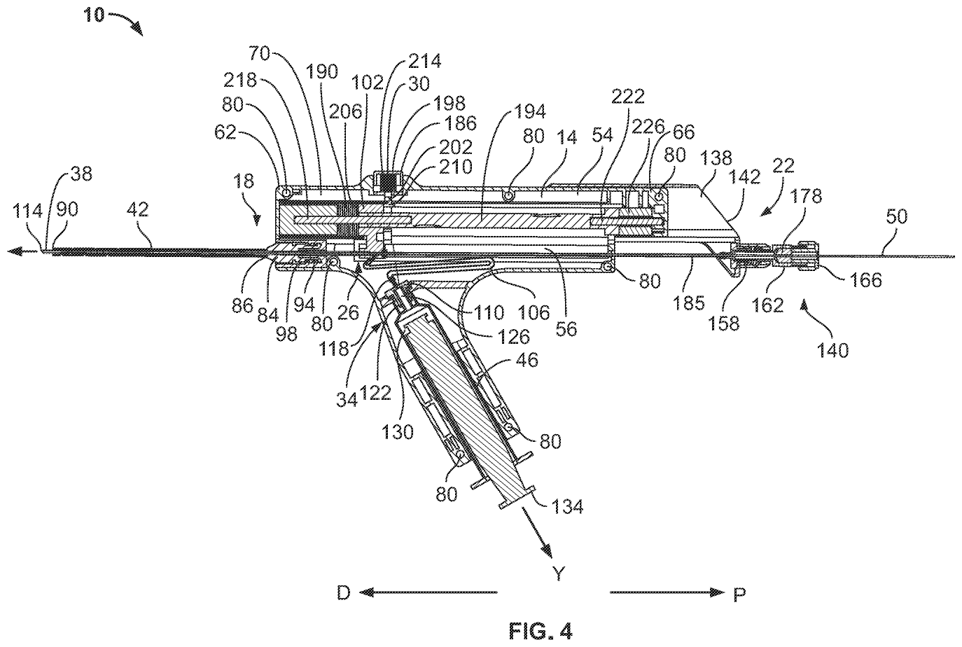

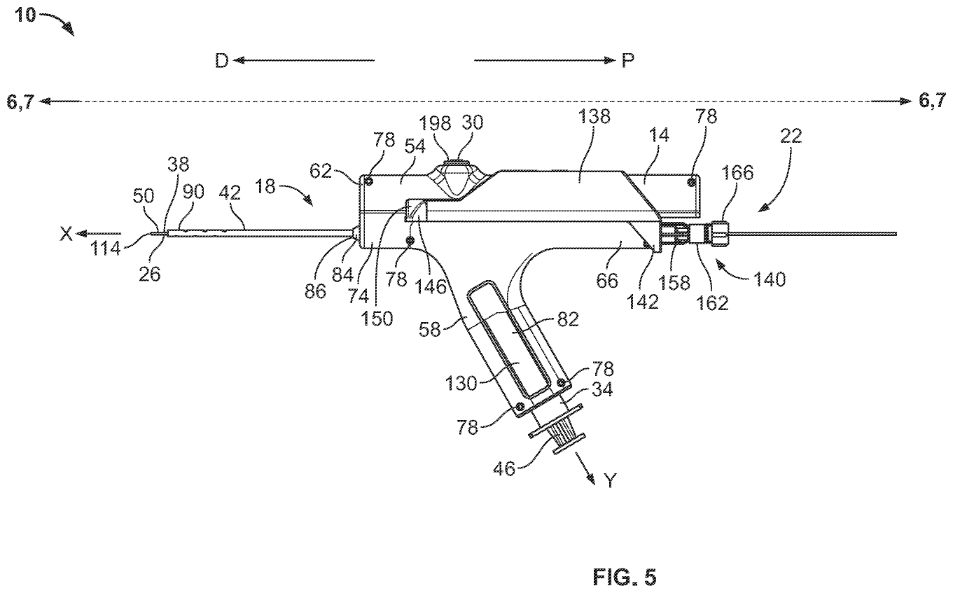

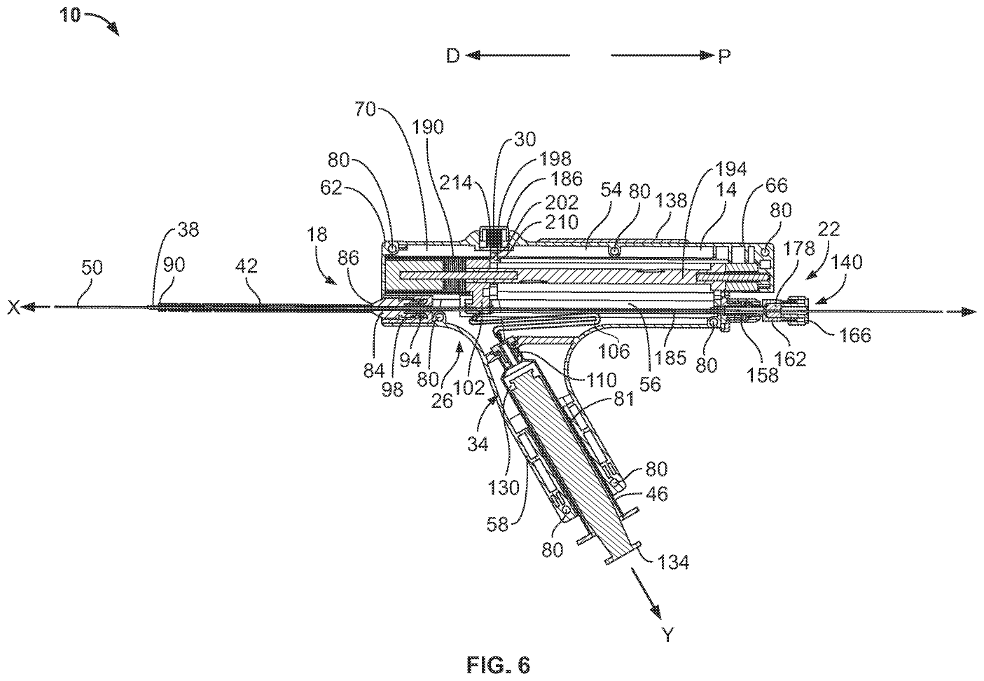

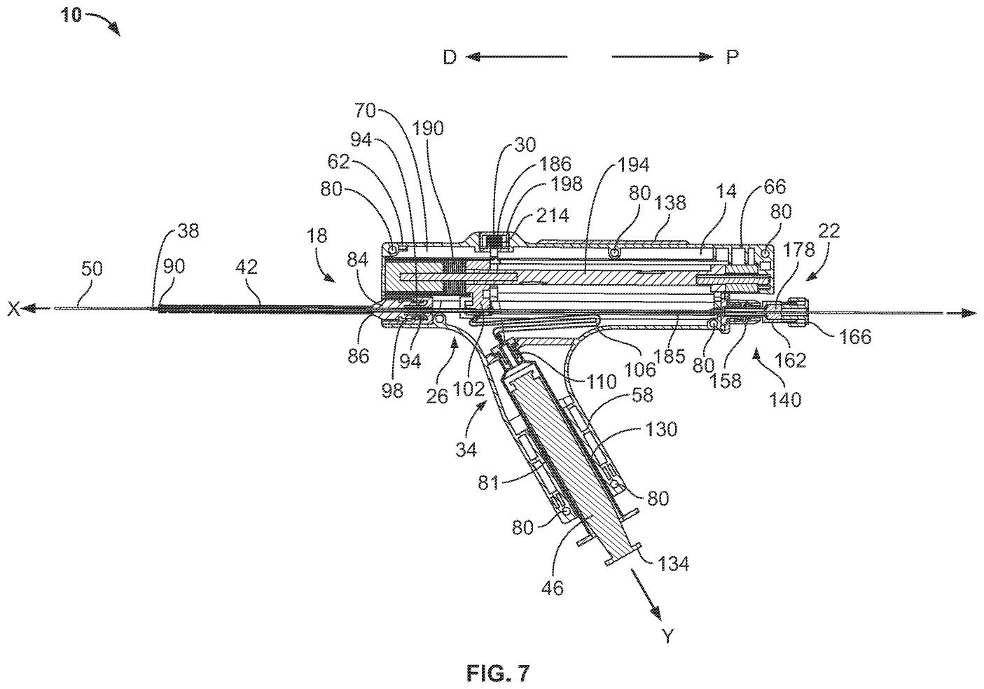

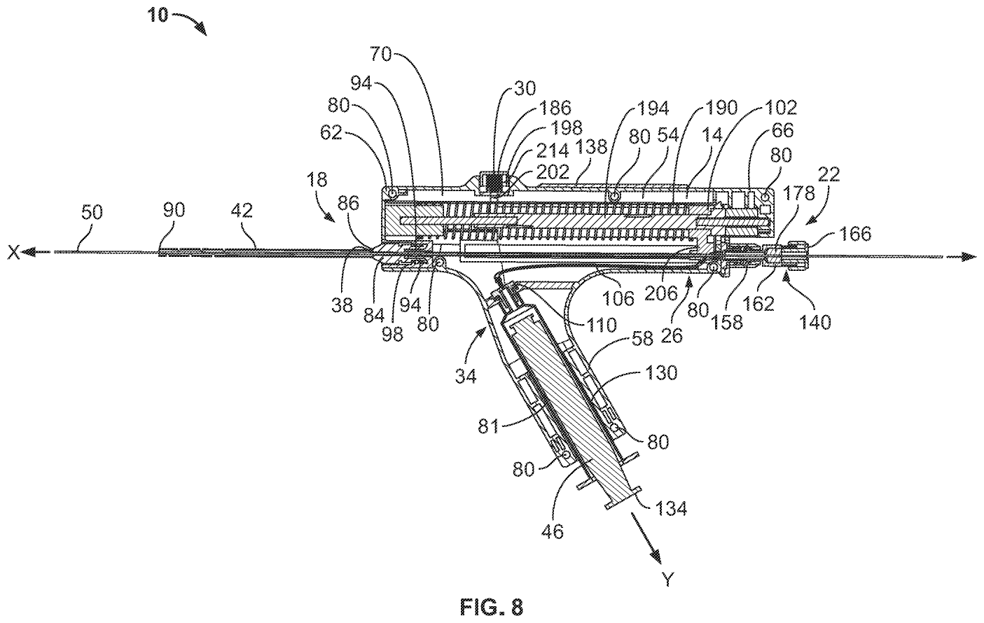

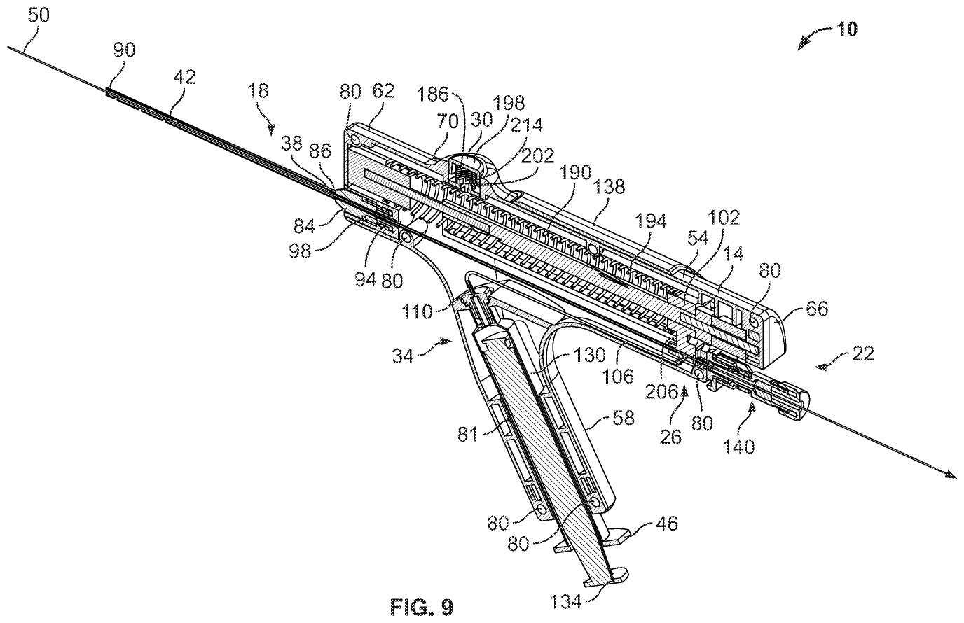

FIGS. 1-9 illustrate a handheld body access device 10 assembled in accordance with the teachings of the present disclosure. The device 10 may be operated to deploy and retract certain components to access a body cavity of a patient, aspirate fluid in the body cavity, and detach a drain port from the device 10 to leave in the patient. FIGS. 1 and 2 illustrate the device 10 in a loaded configuration such that the device 10 is ready for insertion into a patient; FIG. 3 illustrates the internal components of the device 10 in a preloaded configuration; FIG. 4 illustrates the internal components of the device 10 in the loaded configuration; FIGS. 5 and 6 illustrate the device 10 in a guidewire placement configuration; FIG. 7 illustrates the device prior to needle retraction; and FIGS. 8 and 9 illustrate the device 10 with the needle retracted.

Turning first to FIGS. 1 and 2, the device 10 includes a housing 14, a catheter assembly 18, a guidewire assembly 22, a needle assembly 26, an actuation assembly 30, and a syringe assembly 34. The device 10 permits an operator to insert a needle 38 of the needle assembly 26 and a catheter 42 of the catheter assembly 18 into an injection site of a patient, aspirate the injection site by drawing in fluid with a syringe 46 of the syringe assembly 34, slide a guidewire 50 of the guidewire assembly 22 into place, retract the needle 38 from the patient, and detach the catheter 42 from the device housing 14 to leave the catheter 42 in the patient for further drainage.

The housing 14 includes two main components: A barrel 54 and a handle 58 that extends from the barrel 54. The barrel 54 includes a distal end 62, a proximal end 66, and a longitudinal axis X extending between the distal and proximal ends 62, 66 of the barrel 54. The housing 14 has a cavity 56 (FIG. 3) in which the components of the device 10 are disposed, and the housing 14 at least partially encloses the catheter, guidewire, needle, actuation, and syringe assemblies 18, 22, 26, 30, 34. The assemblies 18, 22, 26, 30, 34 are coupled to the housing 14 in a way that permits at least some components of each assembly 18, 22, 26, 30, 34 to extend, protrude, or slide in and out of the cavity 56 of the housing 14. The needle 38, catheter 42, and guidewire 50 are aligned with the longitudinal axis X of the barrel housing 54.

The housing 14 is constructed of a first half 70 and a second half 74 secured together by a plurality of fasteners 78, such as screws, pins, or welds, through bores 80 in each half 70, 74. While the internal detail of the second half 74 is shown, the first half 70 may be identical and/or complementary to the second half 74 to both fit and hold the assemblies 18, 22, 26, 30, and 34 in place. The handle 58 of the housing 14 has a longitudinal axis Y that extends relative to the longitudinal axis X of the barrel 54 at an angle α in a range of approximately 30 degrees to 90. Preferably, the angle α is in a range of 45 degrees to 80 degrees relative to the longitudinal axis X. The handle 58 includes a bore 81 sized to slidably receive the syringe 46, and a window 82 formed in the handle 58 such that an operator may view the contents of the syringe 46 while operating the handheld device 10.

The catheter assembly 18 includes the catheter 42 and a catheter hub 84 removably coupled to the housing 14. In particular, the catheter 42 includes a proximal end 86 coupled to the distal end 62 of the housing 14, and a distal end 90 spaced away from the housing 14. As shown in FIG. 3, the hub 84 includes a proximal end 94 that is removably secured to a luer-type lock fitting 98 formed in the distal end 62 of the barrel 54 of the housing 14. The hub 84 may be removably coupled to the housing 14 by a threaded connection. To remove the catheter 42 from the distal end 62 of the housing 14 and leave the catheter 42 into an injection site of a patient, for example, an operator may rotate the hub 94 about the longitudinal axis X until the hub 94 and catheter disengage from the housing 14. In another example, an operator may pull the hub 94 away from the housing 14 along the longitudinal axis X of the barrel 54 until the end 98 of the hub 94 flexes out of engagement with the fitting 98 of the housing 14. In yet another example, the hub 84 may be coupled to the distal end 62 of the housing 14 by a different locking mechanism such as, for example, a snap-fit connection, a threaded connection, or an interference fit connection.

The needle assembly 18 includes the needle 38, a needle hub 102, and a conduit 106 that fluidly couples a bore of the hollow needle 38 and a syringe adapter 110 of the syringe assembly 34. In FIG. 3, the needle hub 102 is positioned adjacent to the proximal end 66 of the barrel housing 54 in a preloaded configuration and provides fluid communication between the needle 38 and the conduit 106. The needle 38 is retracted into a bore of the catheter 42 such that a distal end or tip 114 of the needle 38 is shielded. In the loaded position, as shown in FIG. 4, the needle hub 102 is adjacent to the distal end 62 of the barrel housing 54 and the distal tip 114 of the needle 38 extends from the distal end 90 of the catheter 42. The needle hub 102 carries both the needle 38 and the conduit 106 and is movable between a first needle position and a second needle position. In the first needle position shown in FIG. 4, the needle 38 extends from the distal end 90 of the catheter 42. In the second needle position shown in both the preloaded position in FIG. 3 and the retracted position in FIGS. 8 and 9, the needle 38 does not extend from the distal end 90 of the catheter 42, and therefore the catheter 42 covers the needle 38. The needle 38 slides within a bore of the catheter 42 as the needle 38 moves along the longitudinal axis X of the barrel 54.

As illustrated in FIG. 3, the syringe adapter 110 includes a port 118 and a female luer fitting 122 that removably couples to a male luer fitting 126 of the syringe 46, as shown in FIG. 4. The syringe assembly 34 is illustrated coupled to the housing 14 of the device 10. The syringe assembly 34 includes the syringe 46 and the syringe adapter 110, and is in fluid connection with the catheter and needle assemblies 18, 26. When the port 118 of the syringe adapter 110 is coupled to the conduit 106 and the female luer fitting 122 of the syringe adapter 110 is coupled to the male luer fitting 126 of the syringe 46, a barrel 130 of the syringe 46 is in fluid connection with the needle 38 and the catheter 42. To couple the syringe 46 to the syringe adapter 110 in the handle 58 of the housing 14, an operator can rotate the syringe in first direction about the longitudinal axis Y of the handle 58 to couple the male luer fitting 126 of the syringe 46 to the female luer fitting 122 of the syringe adapter 110. The operator can rotate the syringe 46 until the syringe 46 is locked into place within the bore 81 of the handle 58. When the syringe 46 is locked to the handle 58, the syringe 46 is secured to the device 10 and may be operated by pulling a plunger 134 of the syringe 46 away from the handle 58. In this position, the barrel 130 of the syringe 46 aligns with the window 82 in the handle 48, as shown in FIGS. 1, 2, and 5, so that an operator may view the contents of the syringe 46 when aspirating a body cavity of a patient. To remove the syringe 46 from the handle 58 of the device 10, the operator may rotate the syringe 46 in a direction opposite from the first direction until the male luer fitting 126 of the syringe 46 decouples from the female luer fitting 122 of the adapter 110.

Turning back to FIG. 3, the syringe adapter 110 is shown alone and without the syringe 46 for clarity. The syringe adapter 110 is secured to the housing 14 and disposed above the bore 81 of the handle 58, and in particular, in an area in which the barrel 54 meets the handle 58. In other examples, the syringe adapter 110 may be located further down the handle 58 or further into the barrel 54 of the housing 14. The syringe adapter 110 is secured to an internal structural component, such as a ribbing, of the housing 14 by a snap-fit connection. However, in other examples, the syringe adapter 110 may be removably coupled to the housing 14 or disposed within the internal cavity 56 of the housing 14 in another manner. For example, instead of being secured to the first half 70 as shown in FIG. 3, the syringe adapter 110 may be secured in place when the first and second halves 70, 74 of the housing 14 are connected.

As shown in FIG. 3, the guidewire assembly 22 includes the guidewire 50, a slide 138 carrying the guidewire 50, and a clamp mechanism 140 coupling the guidewire 50 to the slide 138. The slide 138 is coupled to the barrel 54 of the housing 14 and moves along a track 154 defined in the barrel 54 and between a first guidewire position and a second guidewire position. In the first guidewire position shown in FIGS. 1-4 a portion of the slide 138 is spaced away from the housing 14, and in the second guidewire position shown in FIGS. 5-9, the guidewire 50 extends through the distal end 90 of the catheter 42. In the first guidewire position in FIGS. 1 and 2, a proximal end 142 of the slide 138 is spaced away from the proximal end 66 of the barrel 54. At a distal end 146 of the slide 138, an outward tab 150 extends outward and away from the barrel 54 of the housing 14. The outward tab 150 is positioned, shaped, and sized to comfortably facilitate movement of the slide 138 by an operator's finger while the operator is gripping the handle 58 of the device 10. The slide 138 also includes an inward tab (hidden from view) that is slidably received by the track 154 (FIGS. 1 and 2) formed in the barrel 54 of the housing 14. The inward and outward extending tabs 150 of the slide 138 facilitate operation of the guidewire assembly 22 between the first guidewire position and the second guidewire position.

As illustrated in FIG. 3, the clamp mechanism 140 of the guidewire assembly 22 is a coupled to the proximal end 142 of the slide 138. The clamp mechanism 140 includes a valve 158, such as a Tuohy-Borst adapter valve, a clamp 162, and a cap 166 threadably coupled to the clamp 162. The valve 158 is fixed to the slide 138 such that the slide 138 carries the guidewire and the clamp mechanism 140 as the slide 138 moves along the track 154. The valve 158 seals against the guidewire 50 to prevent aspirated fluid from following the guidewire 50 out of the device 10. The cap 166 and clamp 162 also seal against the guidewire 50 so that the guidewire 50 moves along the longitudinal axis X with movement of the slide 138. In particular, the cap 166 and clamp 162 engage the guidewire 50 in a clamped position, in which the guidewire 50 does not move relative to the slide 138, and an unclamped position, in which the guidewire 50 may move relative to the slide 138. The clamp 162 is fixed to the valve 158 and includes a threaded male fitting 170 that threadably couples to a female fitting 174 of the cap 166. In the clamped position, a ramped stopper 178 disposed in the clamp 162 engages the guidewire 50 against an angled surface 182 of the clamp 162 to fix the guidewire 50 to the slide 138. To unclamp the guidewire 50, an operator may rotate the cap 166 about the longitudinal axis X and relative to the clamp 162 to move the cap 166 in a proximal direction P and release the ramped stopper 178 from engaging the guidewire 50 against the angled surface 182 of the clamp 162. In the unclamped position, an operator may thread the guidewire 50 further into the patient or draw the guidewire 50 out of the patient when the slide 138 is either in the first or second guidewire positions. A hypotube 185 surrounds a portion of the guidewire 50 and extends along a portion the guidewire 50 from a distal tip of the valve 158 and into the needle 38. The hypotube 185 slides freely inside the needle 38 and helps push the guidewire 50 into the patient for approximately two inches of travel.

Turning to FIG. 4, the actuation assembly 30 includes a spring release 186, an actuation spring 190, and drive screw 194. The spring release 186 includes a button 198 that is at least partially disposed outside of the housing 14, and flexible arms 202 that are releasably coupled to the needle hub 102 in the cavity 56 of the housing 14. In the preloaded position, as shown in FIG. 3, the spring 190 is in an expanded configuration and is disposed between the distal end 62 of the barrel 54 and a distal face 206 of the needle hub 102. In the loaded configuration, as shown in FIG. 4, a latch 210 of the needle hub 102 is coupled to the flexible arms 202 of the spring release 186. A spring 214 of the spring release 186 is configured to return the button 198 to the original position when the button 198 is not depressed.

When the button 198 is depressed, as illustrated in FIG. 7, the flexible arms 202 disengage from the latch 210 of the needle hub 102, thereby permitting the actuation spring 190 to move from the compressed position, as shown in FIG. 4, to an expanded position as shown in FIGS. 8 and 9. As the actuation spring 190 expands, the needle hub 102 carries the needle 38 from the first needle position to the second needle position. In other examples, the spring release 186 of the actuation assembly 30 may be a slide or a rotatable knob that is disposed outside of the housing 14 and arranged to release the spring 190 upon activation. In yet another example, a portion of the spring release 186 that is disposed within the housing 14 may be a threaded connection, a lock and key mechanism, or other device that decouples from the needle hub 102 when the spring release 186 is activated. The spring release 186 may be a unitary component or may be composed of multiple components.

Turning back to FIG. 4, the actuation assembly 30 further includes a damper 226 disposed at the proximal end 66 of the barrel 54 and fixed to the drive screw 194 to dampen the force of the actuation spring 194 on the needle hub 102 when the actuation spring 194 is released. The screw 194 includes a course pitch to control the rate of retraction of the needle 38. The needle hub 102 includes a pin (not shown) that couples to the pitch of the screw 194 such that as the needle hub 102 moves in a proximal P direction, the pin causes the screw 194 to rotate. The screw 194 is fixed to a first pin 218 disposed in the distal end 62 of the barrel 54 and a second pin 222 disposed in the proximal end 66 of the barrel 54. The second pin 222 rotates within the damper 226 to dampen the rotation of the screw 194, and therefore the movement of the needle hub 102 in the proximal direction P. In particular, the damper 226 has an inner diameter that rotates with the screw 194 via a connecting member while a rib extending from an outer diameter of the damper 226 is fixed to the housing 14.

To operate the exemplary body cavity access device 10 in a pericardiotomy procedure, for example, an operator holds the handle 58 in the palm of the operator's hand. By gripping the device 10 in this manner, the operator can use their free hand to load the handle 58 with a syringe 46. As previously described, the operator couples the syringe 46 to the device 10 by inserting the syringe 46 into the bore 81 of the handle 58 and rotating the syringe 46 about the longitudinal Y axis relative to the handle 58 until the syringe 46 couples to the syringe adapter 110. During the subsequent steps of the procedure, the operator may hold the device 10, depress the button 198, and push the slide 138 with the same hand while tracing the placement of the needle 38 with an ultrasound wand using the other hand. Operation of the device 10 will now be described with reference to the chronological sequence of FIGS. 4-9.

FIG. 4 illustrates the device 10 loaded and ready for insertion into a body cavity of a patient. In this configuration, a portion of the device 10 may be inserted into the patient to position the needle 38 in the pericardial cavity of a patient. For example, with the aid of an ultrasound wand, the needle 38 may be carefully positioned into the pericardial cavity by piercing the pericardium surrounding the patient's heart. However, in other examples, the device 10 may be used to drain a different body cavity, such as, for example, an abdominal abscess. In FIG. 4, the needle assembly 26 is in the first needle position and the guidewire assembly 22 is in the first guidewire position.

When the device 10 is inserted into a patient, the distal tip 114 of the needle 38 is disposed in the body cavity of the patient. In FIG. 5, the slide 138 of the guidewire assembly 22 has been pushed forward in the distal direction D so that the guidewire 50 extends out through the distal ends 90, 114 of the catheter 42 and needle 38, respectively. To insert the guidewire 50 into the patient, the operator may use their thumb to press against the outward tab 150 of the slide 138 and push the slide 138 in the distal direction D so that the guidewire assembly 22 is in the second guidewire position. In this position, which is illustrated in FIGS. 5 and 6, the operator may aspirate the fluid in the pericardial cavity by pulling the plunger 134 of the syringe 46 and drawing fluid from the pericardial cavity, into the needle 38, through the conduit 106 and syringe adapter 110, and into the barrel 130 of the syringe 46. The contents of the syringe 46 are visible through the window 82 in the handle 58. During fluid aspiration, the needle assembly 26 is in the first needle position and the guidewire assembly 22 is in the second guidewire position. As shown in FIG. 6, the arms 202 of the spring release 186 engage the latch 210 of the needle hub 102.

FIG. 7 illustrates the device 10 the moment the button 198 of the actuation assembly 30 activates, and before the needle assembly 26 retracts. To activate the actuation assembly 30, the operator can press the button 198 downward using an index finger while holding the handle 58 of the device 10 in the same hand. As the operator pushes button 198 downward, the flexible arms 202 of the spring release 186 move away from the needle hub 102 and disengage from the latch 210.

FIGS. 8 and 9 illustrate the device 10 after the needle 38 fully retracts and is in the second needle position. In the needle retracted configuration, the guidewire 50 and the catheter 42 remain inside the patient. The actuation spring 190 is in the expanded position, thereby driving the needle hub 102 from the first needle position to the second needle position such that the needle 38 no longer extends from the distal end 90 of the catheter 42. In this position, the operator unclamps the guidewire 50 from the slide 38 by rotating the cap 166 of the clamp mechanism 140 about the longitudinal axis X, causing the ramped stop 178 of the clamp 162 to disengage from the guidewire 50. The operator can thread the guidewire 50 further into the patient safely and without risk of puncturing the heart with the needle 38. Also in this configuration, the operator can remove the catheter 42 from the distal end 62 of the housing 14 by rotating the hub 84 of the catheter assembly 18 relative to the housing 14 and detach the catheter 42 from the distal end 62 of the barrel 54. Once the catheter 42 is in place in the patient, the operator can withdraw the guidewire 50 from the patient, leaving the catheter 42 in place for further drainage.

The exemplary body cavity device 10 provides all the instrumentation and components to perform a pericardiotomy in the practitioner's hand. The device 10 also permits a single operator to perform a procedure that may require two people to perform. While holding the device 10 in one hand, the operator can insert the needle 38 and catheter 42 into an injection site of a patient, aspirate the injection site by drawing in fluid with the syringe 46, slide the guidewire 50 into place, retract the needle 38 from the patient, and detach the catheter 42 from the device housing 14. To perform a pericardiotomy, for example, the practitioner no longer needs to access each individual instrument laid out on a separate table. Further, the practitioner is free to use their second hand to use an ultrasound while operating the device 10. Further, the device 10 may provide a safer method that involves less risk for threading a guidewire into a patient. Guidewire, which is very thin and slack, is threaded generally using two hands. However, with the needle safely retracted, the operator may single-handedly thread the guidewire without risking injury to the patient.

While the device 10 is described herein as a pericardiotomy device to remove fluid in the pericardium, which surrounds the heart, the disclosed device 10 may be used in other medical procedures to access different body cavities of a patient. The device 10 may be used to drain a cavity of excess fluid, or, in other applications, the device 10 may be used to inject a patient with a medicine via the syringe 46. While the disclosed exemplary device 10 is intended for single-use, in some applications, the device 10 may be sterilized and reused. Additionally, the device 10 may include different fittings or adapters, for example, the syringe adapter, to receive different models or types of syringes.

The figures and description provided herein depict and describe preferred embodiments of handheld medical device for accessing a body cavity for purposes of illustration only. One skilled in the art will readily recognize from the foregoing discussion that alternative embodiments of the components illustrated herein may be employed without departing from the principles described herein. Thus, upon reading this disclosure, those of skill in the art will appreciate still additional alternative structural and functional designs for the handheld body cavity access device. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the methods and components disclosed herein without departing from the spirit and scope defined in the appended claims.

What is claimed:

1. A handheld body cavity access device, the device comprising:
a housing having a barrel, a handle extending from the barrel, and a cavity, the barrel including a distal end, a proximal end, and a longitudinal axis extending between the distal and proximal ends of the barrel;
a catheter including a proximal end coupled to the distal end of the housing, and a distal end spaced away from the housing;
a needle assembly including a needle, a needle hub, and a conduit, the needle hub carrying the needle and the conduit within the cavity of the housing, the needle hub movable between a first needle position, in which a distal end of the needle extends from the distal end of the catheter and a second needle position, in which the distal end of the catheter covers the needle;
a guidewire assembly including a guidewire and a slide carrying the guidewire, the slide is slidably coupled to the barrel of the housing and is movable relative to the housing between a first guidewire position, in which a portion of the slide is spaced away from the housing, and a second guidewire position, in which the guidewire extends through the distal end of the needle; and
a syringe adapter disposed in the cavity of the housing and adapted to couple to a syringe tip of a syringe, the syringe adapter coupled to the conduit of the needle assembly.

2. The device of claim 1, wherein the catheter, needle, and guidewire are aligned with the longitudinal axis of the barrel.

3. The device of claim 1, wherein the handle extends from the barrel at an angle in a range of approximately 30 degrees to 90 degrees relative to the longitudinal axis of the barrel.

4. The device of claim 1, wherein the housing includes a track that is formed in the barrel and is arranged to slidably receive a portion of the slide of the guidewire assembly.

5. The device of claim 1, wherein the housing includes a first half and a second half, the first half including a track to slidably receive a first portion of the slide of the guidewire assembly and the second half including a track to slidably receive a second portion of the slide of the guidewire assembly.

6. The device of claim 1, further including a catheter hub coupled to the catheter and removably coupled to the distal end of housing.

7. The device of claim 1, further comprising a syringe disposed within a bore of the handle and coupled to the syringe adapter, the syringe in fluid communication with the conduit of the needle assembly.

8. The device of claim 1, further comprising an actuation assembly operatively coupled to the needle assembly to move the needle hub between the first needle position and the second needle position.

9. The device of claim 8, wherein the actuation assembly includes a spring and a spring release, the spring release including a first portion disposed outside of the housing and a second portion releasably coupled to the needle hub.

10. The device of claim 9, wherein when the spring release is activated, the second portion of the spring release disengages from the needle hub to permit the spring to move from a compressed position to an expanded position, thereby moving the needle hub from the first needle position to the second needle position.

11. The device of claim 9, wherein the first portion of the spring release is a button and the second portion of the spring release is an arm, the arm adapted to flexibly engage a latch of the needle hub.

12. The device of claim 1, wherein the handle includes a window arranged to align with a window of a syringe barrel.

13. A method of accessing a body cavity using a handheld device, the method comprising:
inserting a needle of a handheld device into a cavity, the handheld device including a housing having a barrel and a handle extending from the barrel, the barrel including a distal end, a proximal end, and a longitudinal axis extending between the distal and proximal ends of the barrel, a catheter including a proximal end coupled to the distal end of the housing and a distal end spaced away from the housing, a guidewire assembly including a guidewire and a slide carrying the guidewire and slidably coupled to the barrel of the housing;
inserting the guidewire through a distal end of the needle by sliding the slide relative to the housing between a first guidewire position, in which a portion of the slide is spaced away from the housing, to a second guidewire position, in which the guidewire extends through the distal end of the needle;
retracting the needle of the handheld device by activating an actuation device; and
inserting the catheter into the cavity.

14. The method of claim 13, wherein retracting the needle includes moving a needle hub carrying the needle within the barrel of the housing between a first needle position, in which the distal end of the needle extends from the distal end of the catheter, to a second needle position, in which the distal end of the needle is drawn into the catheter.

15. The method of claim 13, wherein retracting the needle includes releasing a spring to drive the needle hub from the first needle position to the second needle position.

16. The method of claim 15, further comprising gripping the handle with a hand, wherein inserting the guidewire includes sliding the slide with a thumb of the hand and retracting the needle includes pressing a button with a finger of the same hand.

17. The method of claim 13, further comprising removing the proximal end of the catheter from the distal end of the barrel and leaving the distal end of the catheter inserted in the cavity.

18. The method of claim 13, further comprising drawing fluid from the cavity using a syringe coupled to the handle of the housing, the syringe in fluid communication with the needle.

19. The method of claim 18, further comprising coupling the syringe to a syringe adapter disposed in the housing, the syringe adapter being fluidly coupled the needle via a conduit.

20. The method of claim 18, further comprising inserting the syringe into a bore of the handle of the handheld device.
